(19) United States
(12) Patent Application Publication
(10) Pub. No.: US 2025/0065140 A1
(43) Pub. Date: Feb. 27, 2025

(54) MAGNETIC STIMULATION COIL ALIGNMENT APPARATUS

(71) Applicant: The United States Government as represented by the Department of Veterans Affairs, Washington, DC (US)

(72) Inventor: Punit Vaidya, Cleveland, OH (US)

(21) Appl. No.: 18/724,999

(22) PCT Filed: Jan. 3, 2023
(86) PCT No.: PCT/US2023/010033

§ 371 Date: Jun. 27, 2024

Related U.S. Application Data
(60) Provisional application No. 63/295,619, filed on Dec. 31, 2021.

(51) Int. Cl. A61N 2/02; A61N 2/00

(52) U.S. Cl. CPC A61N 2/02; A61N 2/006

(57) ABSTRACT

An apparatus comprises a magnetic stimulation coil having a central axis and one or more capacitive sensors. The one or more capacitive sensors can be configured to contact a target and/or a target area and/or a target surface. Processing circuitry can be configured to detect the contact between at least one capacitive sensor and, for example, the target surface. One or more range sensors can be spaced from the central axis of the magnetic stimulation coil. A display can be configured to display a location corresponding to the contact between the at least one capacitive sensor and the target area, a distance between at least one range sensor and the target surface, and a rotation of a coil with respect to a reference angle.

### CROSS-REFERENCE TO RELATED APPLICATIONS

This application claims priority benefit of U.S. Provisional Application No. 63/295,619, filed Dec. 31, 2021, the entirety of which is incorporated herein by reference.

### BACKGROUND

Magnetic Stimulation (MS) is a non-invasive method of neuronal stimulation in which rapidly changing magnetic fields generated by an electromagnetic coil induce electric current in nervous tissue under the coil (such as in the cortex or a peripheral nerve). MS can be used for diagnostic or therapeutic purposes. In the case of transcranial magnetic stimulation (TMS), the coil is placed over targeted areas of the cortex. Single pulses of TMS can produce action potentials in neurons under the coil. When pulses are applied repetitively as in repeated TMS (rTMS), the technique can be used to induce longer-lasting effects on neuronal excitability. Depending on the frequency and pattern of pulses, rTMS can have an excitatory or inhibitory effect. A common clinical use of rTMS is for the treatment of medication resistant major depressive disorder. Because the induced electrical activity from TMS pulses is highly focused and decays very rapidly with distance from the center of the coil, proper contact with the target and coil orientation in 3-dimensional space with respect to the patient's head are important for optimal stimulation. Several independent aspects of coil positioning can influence efficiency of stimulation. For example, even small coil-to-head gaps can result in marked reduction of stimulation intensity at the cortex, and by consequence, can reduce clinical efficacy. Similarly, the MS coil should ideally be tangential to the patient's scalp. If the MS coil is making contact with the scalp but not centered directly over the desired target and in a tangential orientation, efficacy may also be compromised. Additionally, the angle of the coil, with respect to the mid-sagittal plane of the head, can influence cortical response to TMS. An angle of 45 degrees is generally considered optimal for cortical stimulation.

Current solutions include neuronavigation, which relies on a complex imaging and computational system with multiple cameras and optical trackers which are attached to the coil and the patient's head. The system creates a 3-dimensional reconstruction of the coil location and orientation over an MRI image of the patient's brain or a generic head model. Such a technique is typically reserved for research purposes, as it is costly, time-intensive, and not practical for routine clinical application. Thus, in clinical practice, visual approximation is most commonly used to place the MS coil and is the technique employed in most of the devices that have been FDA approved for the treatment of major depression. This method is crude and imprecise because it does not provide objective confirmation of proper coil placement or contact with the target. This method is also prone to errors, and can compromise clinical efficacy. Many of the methods for MS coil placement rely on a cap, resembling a swimmer's cap, which is fit snuggly over the patient's head. The target is marked on the cap, and the coil is centered over the target at the start of each treatment. However, due to the dimensions of the coil, once placed on the head, the target marking and center of the underside of the coil are occluded from visual inspection, so the location of the edge of the coil is marked on the cap. As the coil is flat, the projection of this edge on the curved surface of the head is prone to errors from visual parallax, both at times of target demarcation and each time the coil is placed for subsequent treatments. Additionally, one must rely on visual estimation to determine if the coil is tangentially placed on the scalp and at 45 degrees from the mid-sagittal plane. All of these potential sources of error can result in significant variability in coil placement between TMS operators and over repeated treatment session and, thus, may result in suboptimal therapeutic response.

For the treatment of major depression, the MS coil must be properly positioned for the duration of the treatment, which typically lasts from a few minutes to an hour. Conventional devices do not provide continuous feedback to the operator regarding proper coil placement. Small movements of the patient's head (for example, by head turning, coughing, sneezing, or shifting in the treatment chair) may change the coil-to-target placement and thereby impact clinical efficacy. Thus, it is important that feedback on coil positioning is provided to the TMS operator not only prior to the start of each treatment session, but continuously over the duration of each treatment session.

### SUMMARY

Described herein, in various aspects, are methods, an apparatus, and systems configured for magnetic stimulation. The apparatus may comprise a coil having a central axis and one or more capacitive sensors, wherein the one or more capacitive sensors may be configured to contact a target area of a target surface. The apparatus may comprise processing circuitry configured to detect an engagement between the target area and at least one capacitive sensor of one or more capacitive sensors. The apparatus may comprise a plurality of range sensors. In a non-limiting embodiment, the plurality of range sensors may be spaced from the central axis of the magnetic stimulation coil. Either or both of the apparatus and the system may comprise a display. The display may be configured to display a location corresponding to the engagement between the target area and the at least one capacitive sensor, and a distance between each range sensor and the target surface.

The apparatus may further comprise a first light emitting device configured to display a first line, and a second light emitting device configured to display a second line. Each of the first light emitting device and the second light emitting device may comprise a laser diode.

The apparatus may further comprise at least one orientation sensor, wherein the processing circuitry is configured to determine an orientation of the magnetic stimulation coil apparatus based on data from the at least one orientation sensor. The at least one orientation sensor may comprise a three-axis accelerometer, a three-axis gyroscope, a three-axis magnetometer, combinations thereof, and the like.

The apparatus may further comprise memory. The processing circuitry may be configured to store in the memory a reference orientation based on at least one measurement from the at least one orientation sensor. The processing circuitry may be configured to determine a relative orientation of the MS coil with respect to the reference orientation. The display may be further configured to display the relative orientation of the MS coil apparatus with respect to the reference orientation.

Each capacitive sensor may be evenly spaced from each respective adjacent capacitive sensor. The apparatus can further comprise a wireless transmitter that is configured to transmit data captured by the apparatus to a receiver operably coupled to a remote display.

The one or more capacitive sensors may be centered with respect to the central axis, and the plurality of range sensors may be equally spaced from the central axis. The one or more capacitive sensors may be configured in one or more rows or columns.

The apparatus may further comprise a second orientation sensor, wherein the processing circuitry is configured to compare orientation data from the second orientation sensor to orientation data from the at least one orientation sensor.

The apparatus may comprise a housing configured to couple to a magnetic stimulation coil device and one or more capacitive sensors, wherein the one or more capacitive sensors is configured to contact a target area of a target surface. The processing circuitry may be configured to detect an engagement between the conductor and at least one capacitive sensor. The plurality of range sensors may be disposed around a circumference of the one or more capacitive sensors.

The apparatus can further comprise a display that is configured to display: a location corresponding to an engagement between the conductor and at least one capacitive sensor of the one or more capacitive sensors, and a distance between each range sensor of the plurality of range sensors and the target surface.

The apparatus can further comprise at least one orientation sensor, wherein the processing circuitry is configured to determine an orientation of the magnetic stimulation coil apparatus based on data from the at least one orientation sensor. The processing circuitry may be configured to store in the memory a reference orientation based on at least one measurement from the at least one orientation sensor. The processor may be configured to determine a relative orientation of the MS coil with respect to the reference orientation. The display may be further configured to display the relative orientation of the MS coil apparatus with respect to the reference orientation.

The apparatus may further comprise a wireless transmitter that is configured to transmit data captured from the apparatus to a receiver operably coupled to a remote display.

A system may comprise an apparatus comprising a magnetic stimulation coil having a central axis and one or more capacitive sensors, wherein the one or more capacitive sensors is configured to contact the conductor on the target area of the target surface. Processing circuitry may be configured to detect the engagement between the conductor and the at least one capacitive sensor of the one or more capacitive sensors. The plurality of range sensors may be spaced from the central axis of the magnetic stimulation coil. The apparatus can further comprise wireless transmitter. The system can further comprise a remote display comprising a receiver. The remote display may be configured to display a location corresponding to the engagement between the conductor and the one or more capacitive sensors and a distance between each range sensor of the plurality of range sensors and the target surface. The wireless transmitter may be configured to transmit data captured by the apparatus to the receiver of the remote display.

The system can further comprise at least one orientation sensor. The processing circuitry may be configured to determine an orientation of the magnetic stimulation coil apparatus based on data from the at least one orientation sensor. The remote display can comprise memory. The processing circuitry may be configured to store a reference orientation based on at least one measurement from the at least one orientation sensor. The processor may be configured to determine a relative orientation of the MS coil with respect to the reference orientation. The display may be further configured to display the relative orientation of the MS coil apparatus with respect to the reference orientation.

A method may comprise: receiving a signal corresponding to an engagement between the target area and at least one capacitive sensor of one or more capacitive sensors; determining, based on the signal, a contact location, wherein the contact location is a position between the first capacitive sensor and the second capacitive sensor; receiving a distance measurement from at least one range sensor of one or more range sensors; displaying the contact location on a display; and displaying the distance measurement from the at least one range sensor on the display.

Displaying the contact location on the display can comprise graphically displaying the contact location as a radial offset from a center point. Displaying the contact location on the display can comprise identifying and displaying which of the capacitive touch sensors in in contact with the target area of the target surface. For example, the display may be configured to output a virtual representation of the one or more capacitive touch sensors and highlight one or more sensors presently contacting the target area of the target surface.

Displaying the distance measurement from each of the plurality of range sensors on the display can comprise graphically displaying the distance measurement from each of the plurality of range sensors as a radius from a center point, a graph, chart, table, or any other graphic.

The method can further comprise calculating a vector as a function of each distance measurement from each of the plurality of range sensors and displaying the vector on the display.

The method can further comprise transmitting the signal and the contact location to the display, wherein the display is a remote display.

Additional advantages of the invention will be set forth in part in the description that follows, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIGS. 1A-1C show example apparatuses;

FIG. 2 shows an example system;

FIG. 3 shows an example apparatus;

FIG. 4 shows example diagrams;

FIG. 5 shows an example apparatus;

FIGS. 6A-6B show example images of a cap and a target marker for use with an MS coil apparatus;

FIGS. 7A-7C show example displays;

FIGS. 8A-8B show example displays;

FIGS. 9A-9C show example displays;

FIGS. 10A-10D show example images showing use cases;

FIG. 11 shows an example schematic;

FIG. 12 shows an example remote display;

FIG. 13 shows an example schematic; and

FIG. 14 shows an example system.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes-from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, flash drive, SD card or similar non-volatile memory card, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded onto a microcontroller, general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a microcontroller, computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems or one or more microcontrollers that perform the specified functions or steps, or combinations of special purpose hardware and computer instructions.

Hereinafter, various embodiments of the present disclosure will be described with reference to the accompanying drawings. As used herein, the term “user” may indicate a person who uses an electronic device or a device (e.g., an artificial intelligence electronic device) that uses an electronic device.

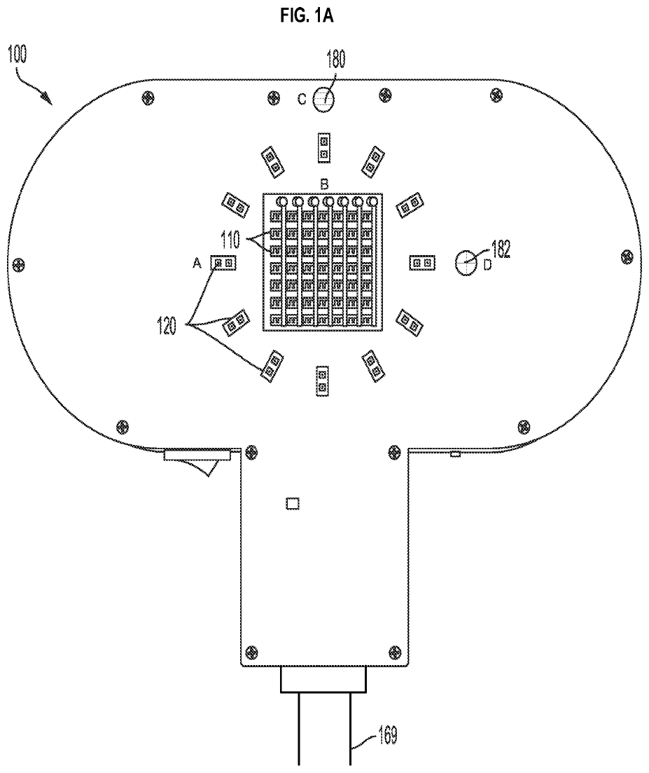

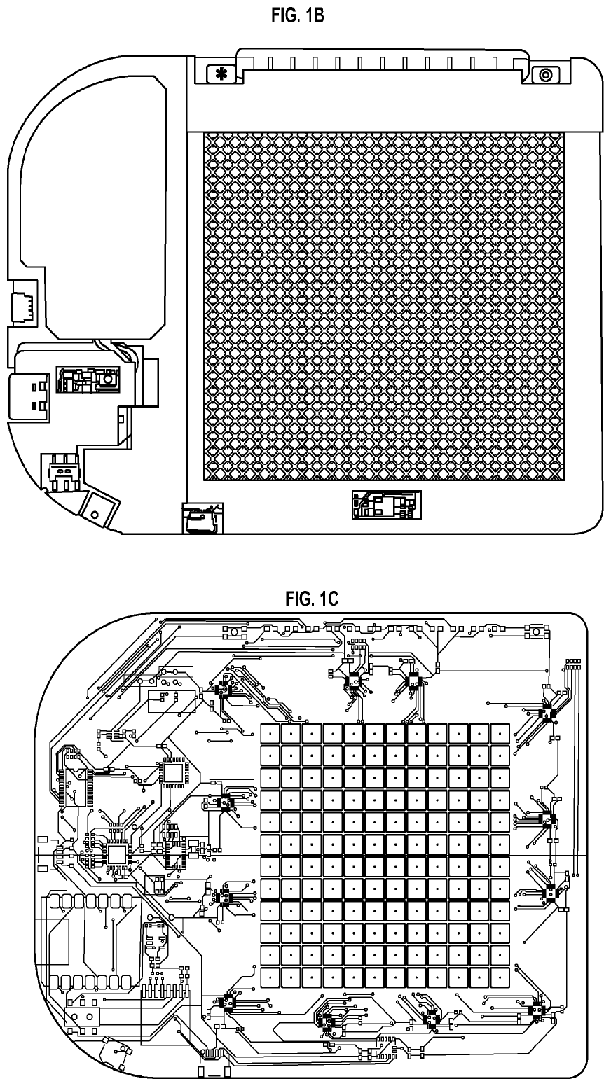

FIG. 1A shows an example MS coil apparatus 100. In some embodiments, the MS coil apparatus 100 can comprise a figure-eight MS coil. In further embodiments, the MS coil apparatus 100 can comprise a circular coil, which can be beneficial in stimulating peripheral nerves. In still further embodiments, the MS coil apparatus 100 can comprise various other coil patterns, such as an h-coil pattern. The MS coil can be configured to generate an electromagnetic field along an axis. The MS coil may be configured to selectively stimulate a target area of a patient's body. The MS coil (and MS coil apparatus 100) may be coupled, for example via a cable 169 to a pulse generator and/or stimulator (not shown). For example, the MS coil may be configured for TMS and/or rTMS. Transcranial magnetic stimulation (TMS) is a noninvasive procedure that uses magnetic fields to stimulate nerve cells in the brain to improve symptoms of depression. During an rTMS session for depression, an electromagnetic coil is placed against a patient's scalp. The electromagnet painlessly delivers a magnetic pulse that stimulates nerve cells in the region of the brain thought to be involved in mood regulation. This therapy me activate one or more regions of the brain that, in a patient with depression, exhibit decreased activity. The MS coil may be configured for other treatments directed towards other areas of the patient's body. For example, the MS coil may be configured for transcutaneous magnetic spinal cord stimulation, wherein the MS coil may be applied in proximity to a patient's spine.

The MS coil apparatus 100 may comprise a system configured to tangentially align the MS coil apparatus 100 with respect to the patient's head. The MS coil apparatus 100 may comprise a sensing module. For example, the sensing module may comprise a plurality of range sensors 120 and/or one or more capacitive sensors 110. The plurality of range sensors can be spaced from the central axis of the MS coil. The MS coil apparatus 100 can comprise the plurality of range sensors 120 spaced in a circular pattern that is centered about an axis of the MS coil. The plurality of range sensors may comprise either or both of distance sensors and/or proximity sensors. The plurality of range sensors 120 can be time-of-flight sensors that can send laser pulses and measure the time for each pulse to reflect off a surface and return to a detector. Other methods of range sensing, such as ultrasonic range sensing, light detection and ranging (LiDAR), infrared range sensing, combinations thereof, and the like, may also be implemented.

The plurality of range sensors 120 may be equally spaced from the axis of the MS coil. Each range sensor of the plurality of range sensors 120 may be configured to detect a respective distance to the patient's head. The plurality of range sensors 120 may be configured to determine one or more distance measurements. The MS apparatus 100 may be configured to represent the one or more distance measurements as one or more vectors having the measured distance as the vector's magnitude and an angular value defined as an azimuthal angle of the range sensor about the MS coil's axis. The MS coil apparatus 100 can be considered tangentially oriented with respect to a patient's head when the sum of all of the vectors is or is about zero.

The MS coil apparatus 100 can comprise one or more capacitive sensors 110. In some embodiments, the one or more capacitive sensors 110 may be configured in a 7×7, 9×9, 12×12, or 15×15 grid pattern. The one or more capacitive sensors 110 may be configured to cover an area, for example a two inch by two inch square area. The one or more capacitive sensors 110 can be centered with the axis.

For example, the capacitive sensor may comprise a first conductive element, and a second conductive element. The first and second elements may be electrically insulated from each other. The first and second elements may have any form. One or both elements may be conductive structures consisting of several parts. Electric ground or earth may also be used as a conductive element of the first or second conductive elements. Each of the first and second conductive elements may have a respective connecting terminal.

The first and second elements may be plates. The plates may be disposed in or on an electrically insulating substrate. The plates may form a capacitive system together with the medium located between said plates. The capacitive system may a capacitance value (CX). For simplicity, the symbol CX is herein used to refer to the physical entity (capacitor) as well as to the measurable quantity (capacitance).

The presence of an object (e.g., the patient's head and/or a target disposed on a cap thereon) in the vicinity of the capacitive sensor changes the dielectric permittivity of the medium between the plates. Thus, the presence of the object changes the capacitance CX, when compared with a situation when the object is far away from the sensor. When the change in capacitance CX satisfies a threshold, the particular capacitive sensor can be interpreted as making appropriate contact with the target.

The one or more capacitive sensors 110 may be configured for either or both of self-capacitance and/or mutual touch capacitance. For example, in a self-capacitance configuration (as shown in FIG. 1B), the one or more capacitive sensors 110 may be configured to measure changes in capacitance with respect to earth ground. For example, considering a parallel-plate configuration, the electrode forms one plate of a capacitor, with the other plate being either ground or the user's finger or a target. A touch causes the electrode capacitance to increase, as the human body “adds” capacitance to that of the system. Self-capacitive measurement may use one or more electrodes to measures the change in capacitance with respect to ground caused by a typical user's touch; the target results in a higher capacitance compared to the baseline measured value. Any parasitic capacitances to ground in the system may be minimized. Further, self-capacitive electrodes project electric field lines in all directions and thus, an interaction may occur (and be detected) on both sides of the electrode. In the self-capacitance configuration, sensors may be arranged in an X-Y grid wherein the columns and rows operate independently. With self-capacitance, current senses the capacitive load of a finger on each column or row. This produces a stronger signal than mutual capacitance sensing. The self-capacitance configuration shown in FIG. 1B comprises 23 columns and 24 rows of electrodes. The grid in FIG. 1B could be used for mutual cap touch sensing as well (similar grids are used in touchscreens on phones/tablets, and capable of multitouch because they use mutual cap touch sensing technique). The columns are distinguished by holes for the vias in the PCB. In that example, the target marker on the patient's cap may be configured to make contact with 2 electrodes (column and row) so the device may determine a location of the coil with respect to the target. Additionally and/or alternatively, each pad may be a separate electrode of a capacitive touch sensor.

For example, in a mutual-touch configuration (as shown in FIG. 1C), the one or more capacitive sensors 110 may be configured with two electrodes that together represent the two plates of the capacitor. An interaction (e.g., contact with the target), modifies the field between the two electrodes and reduces the capacitive coupling between the electrodes. Mutual capacitive sensors may have a capacitor at each intersection of each row and each column. For example, a 12-by-16 array would have 192 independent capacitors. A voltage is applied to the rows or columns. Bringing the target, a finger or conductive stylus near the surface of the sensor changes the local electric field which reduces the mutual capacitance. The capacitance change at one or more individual points on the grid can be measured to accurately determine the touch location by measuring the voltage in the other axis. Mutual capacitance allows multi-touch operation where multiple targets, fingers, palms or styli can be accurately tracked at the same time. In the mutual capacitance configuration, the target marker may be configured to make contact with one or more of the pads/capacitive touch electrodes so the microcontroller may determine a location of the target with respect to the coil.

According to some aspects, the MS coil apparatus 100 may comprise a vertical axis laser 180 and a horizontal axis laser 182, one or more of which may comprise a laser diode. The vertical axis laser 180 may emit a first laser line that is aligned with the MS coil's axis, and the horizontal laser 182 may emit a second laser line that is perpendicular to the first laser line and is aligned with the MS coil's axis. Accordingly, the first and second laser lines can intersect at the stimulation site. The lasers lines can optionally be turned on and off.

The MS coil apparatus 100 may comprise a rechargeable battery, microcontroller, LED indicators, piezo buzzer, speaker, orientation sensor, wireless transceiver, switches, and one or more capacitive touch sensor pads in the center. The one or more capacitive touch sensor pads may comprise copper on a flexible or rigid PCB substrate. The touch sensor electrodes may comprise indium tin oxide on glass or plastic or some other conductive material rather than copper. The MS coil apparatus 100 may comprise 2 or more pairs of range sensors located opposite of each other configured to determine if the coil is tangential to the target surface.

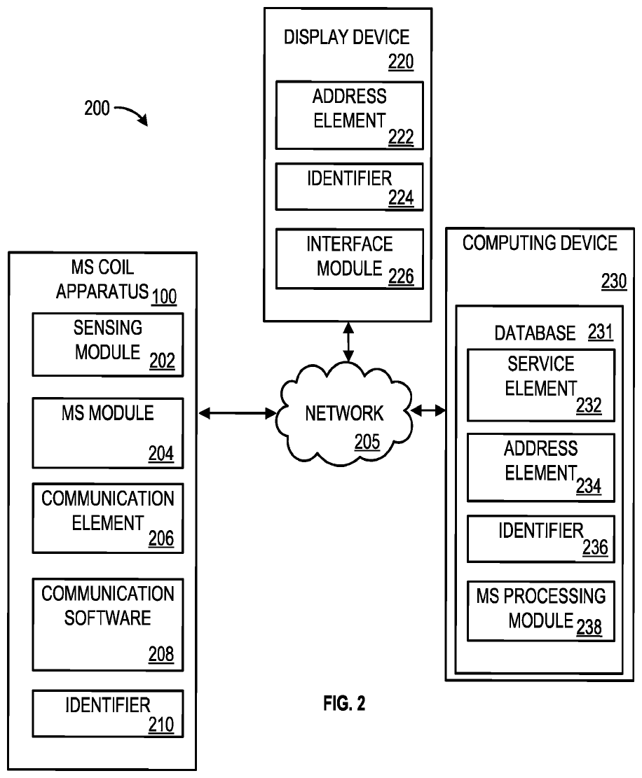

FIG. 2 shows an example system 200 comprising the MS coil apparatus 100, a network 205, a display device 220, and computing device 230. The MS coil apparatus may comprise a sensing module 202 a MS module 204 a communication element 206, communication software 208 and an identifier 210. The MS module may comprise the MS coil.

The sensing module 202 may comprise the plurality of range sensors and/or the one or more capacitive sensors 110. The plurality of range sensors 120 can be spaced from the central axis of the MS coil. The plurality of range sensors 120 may be spaced in a circular pattern that is centered about the axis of the MS coil. The plurality of range sensors 120 may be time-of-flight sensors configured to send laser pulses and measure the time for each pulse to reflect off a surface and return to a detector. Other methods of range sensing can be used such as triangulation sensors and phase modulation sensors. The plurality of range sensors 120 may be equally spaced from the axis of the MS coil. One or more range sensors of the plurality of range sensors 120 may be configured to detect one or more respective distances between the patient's head (e.g., the target area of the target surface) and the plurality of range sensors. The plurality of range sensors 120 may be configured to determine one or more distance measurements. The MS coil apparatus 100 may be configured to represent the one or more distance measurements as one or more vectors. The one or more vectors may include the measured distance as the vector's magnitude and an angular value defined as an azimuthal angle of the range sensor about the MS coil's axis. The MS coil apparatus 100 can be considered tangentially oriented with respect to a patient's head when the sum of all of the vectors is zero.

The MS module 204 may comprise a figure-eight MS coil. In further embodiments, the MS module 204 can comprise a circular coil, which can be beneficial in stimulating peripheral nerves. In still further embodiments, the MS module 204 can comprise various other coil patterns, such as an h-coil pattern. The MS coil can be configured to generate an electromagnetic field along an axis to selectively stimulate a target area of a patient's brain or peripheral nerve.

The communication element 206 may be configured to provide an interface configured to interact with the MS coil apparatus 100 and/or any other device/component of the system 100. The communication element 206 may be any interface for presenting and/or receiving information to/from the user, such as information associated with one or more MS treatment regiments, and/or the like. The communication element 206 may request or query various files from a local source and/or a remote source. The communication element 206 may transmit data to a local or remote device, such as and/or any device/component of the system 100. The communication element 206 may include a communication interface such as a web browser (e.g., Internet Explorer®, Mozilla Firefox®, Google Chrome®, Safari®, or the like). Other software, hardware, and/or interfaces may be used to provide communication between the user and one or more of the devices of system 101, such as network 205 (e.g., Wi-Fi network, Internet, a private network, a public network, a content delivery network, cellular network, etc.), and any other device/component of the system 100.

The communication software 208 may be any combination of firmware, software, and/or hardware. The communication software 208 may facilitate the MS coil apparatus 100 communicating with the display device 220 and/or the computing device 230. For example, the communication software 208 may facilitate the MS coil apparatus 100 communicating with the network 205.

The identifier 210 may comprise an address element and a service element. The address element may comprise a network address (e.g., an IP address, etc.), a media access control (MAC) address, or the like. The address element may be used to establish a communication session between the MS coil apparatus 100 and the network 205, other devices and/or components of the system 101, and/or the like. The address element may be used as an identifier and/or locator of the MS coil apparatus 100. The address element may be persistent for a particular network (e.g., network 205).

The network 205 may comprise any communications network such as, for example, an optical fiber network, a coaxial cable network, a hybrid fiber-coaxial cable network, a wireless network, a satellite system, a direct broadcast system, or any combination thereof.

The display device 220 may comprise an address element 222, an identifier 224, and an interface module 226. The interface module 226 may comprise a monitor, an LCD (Liquid Crystal Display), light-emitting diode (LED) display, television, smart lens, smart glass, and/or a projector. The display device 220 may be configured to receive data from either or both of the MS coil apparatus 100 or the computing device 230. The display device 220 may render a user interface (UI). The UI may be part of an application, such as a mobile application executed on the user device 220. The mobile application may be used to output the data generated by the computing device 230, for example. The UI may include a communication interface such as a web browser (e.g., Internet Explorer®, Mozilla Firefox®, Google Chrome®, Safari®, or the like), media player, application (e.g., web application, mobile application, media device application), and/or the like. The UI may be used to specify a target audience segment such that the data may be generated for the specified target audience segment (e.g., to rank time slots or content networks/items by index parameter or likelihood of having viewers clustered into a target audience subset corresponding to the specified target audience segment).

The address element 222 may comprise or provide an internet protocol address, a network address, a media access control (MAC) address, an Internet address, and/or the like. The address element 222 may be relied upon to establish a communication session between the MS coil apparatus 100, the display device 220, the computing device 230, and/or other devices and/or networks. The address element 222 and the identifier 224 may be stored in the database 231. The address element 222 may be used as an identifier or locator of the computing device 230. The address element 222 may be persistent for a particular network. The address element 222 may be used to identify or retrieve data. The computing device 230 may provide or enable data flow to the display device 220, such as information MS treatment regimens or the like.

The computing device 230 may comprise a database 231, a service element 232, an address element 234, an identifier 236, and an MS processing module 238.

The database 231 may be associated with an identifier 236. The identifier 236 may be any identifier, token, character, string, or the like, for differentiating the computing device 230 from other devices such as another computing server. The identifier may be dynamic, static, temporary, and/or persist for a specified or unspecified time. Other information may be represented by the identifier 236. The identifier 236 may comprise the address element 234.

The service element 232 may comprise an identification of a service provider associated with the computing device 230 and/or with the class of the computing device 230, with the MS coil apparatus 100 and/or with the class of the MS coil apparatus 100. The class of the computing device 230 may be related to a type of device, a device capability, type of service being provided, and/or a level of service (e.g., business class, service tier, service package, etc.). The service element 232 may comprise information associated with a communication service provider (e.g., Internet service provider) that is providing or enabling data flow such as communication services to the computing device 230. The service element 232 may comprise information relating to a preferred service provider for one or more particular services relating to the computing device 230 and/or the MS coil apparatus 100.

The address element 234 may comprise or provide an internet protocol address, a network address, a media access control (MAC) address, an Internet address, and/or the like. The address element 234 may be relied upon to establish a communication session between the MS coil apparatus 100, the display device 220, the computing device 230, and/or other devices and/or networks. The address element 234 and the identifier 236 may be stored in the database 231. The address element 234 may be used as an identifier or locator of the computing device 230. The address element 234 may be persistent for a particular network. The address element 234 may be used to identify or retrieve data. The computing device 230 may provide or enable data flow to the display device 220, such as information regarding MS treatment regiments or the like.

The MS processing module 238 may be configured to receive MS data from the MS coil apparatus 100 and process the MS data. The MS data may comprise data associated with one or more MS treatment regimens and/or MS coil apparatus alignment data. For example, the computing device 230 may be configured to receive, from the MS coil apparatus 100, data gathered by the plurality of range sensors and/or the one or more capacitive sensors 110. The MS processing module may receive the MS data and may process the MS data so as to send the MS data to the display device 220 for output as described further herein.

For example, the MS processing module 238 may be configured to determine a treatment score. The treatment score may indicate, in a variety of senses, whether or not (or a degree to which), a treatment was optimally administered (e.g., a treatment quality score). The treatment quality score may indicate a quality of the treatment session. The treatment quality score may indicate a time-weighted average of the quality of the MS treatment administration.

The MS processing module 238 may receive one or more optimal treatment quality parameters. The optimal treatment quality parameters may indicate for example, an optimal timing parameter such as an optimal treatment duration and/or an optimal pulse train duration, an optimal voltage, an optimal alignment (e.g., with respect to, for example, the sagittal plane or some other target area), combinations thereof, and the like. The optimal treatment quality parameters may be determined, for example, during calibration of the MS coil apparatus. For example, a calibration block lined with the same material used in the target marker sticker may be applied to the one or more capacitive sensors, so that it can be used to set the threshold value of the raw capacitive touch sensor reading at each position to determine “contact”. For example, a conductive felt may be used for the target marker material. The conductive felt may produce a stronger signal on the cap touch sensors than touch with a fingertip, so a target marker of this material could be placed directly on the skin and be distinguished from touch.

A treatment image may be created with the raw capacitive sensor readings at each x/y location of the grid (e.g., xₙ, yₙ). If properly implemented, and with low noise, it may be determined if the coil is positioned tangentially with the target, with or without the use of range sensors. This may be achieved by creating a weighted average of all the raw sensor readings and seeing and comparing the weighted average of all the raw sensor readings with a location of the maximum capacitive touch reading. Deviation may be determined and thus the degree of tilt deviation with respect to tangential placement may be determined.

During the course of treatment, via one or more of the devices or components described herein, such as the range sensors and alignment sensors, the MS processing module 238 may receive alignment data and pulse data. For example, the MS processing module 238 may receive a measured timing parameter such as a measured treatment duration and/or a measured pulse train duration, a measured alignment, combinations thereof, and the like. The MS processing module 238 may be configured to compare the optimal treatment parameters with the measured parameters and determine the treatment quality score.

For example, in the case of rTMS for depression, an optimal alignment might be defined as: pulses administered with the center of the coil touching the target marker (as detected by a capacitive sensor), with coil positioned tangentially to the surface of the head and rotated 45 degrees from the mid-sagittal line would be considered optimal (e.g., if using a numerical scale from 0-100, such positioning during a pulse could be calculated as “100”). Deviations from this optimal position would result in poorer quality treatment and reduction from the score. In the event that the coil is not centered over the target, but otherwise appropriately positioned, the score could be reduced proportional to the deviation of the center of the coil from the target. Off-tangent tilt or rotation of the coil beyond a certain threshold from 45 degrees could also reduce the score. Since the magnetic field dissipates rapidly in the z-axis away from the coil, if one of the capacitive sensors is not touching the target, the score could be “0” for that pulse. The current and time-weighted average quality score could be displayed during the treatment and at the end to give the MS operator and patient some assessment of the adequacy of the MS coil positioning for the treatment.

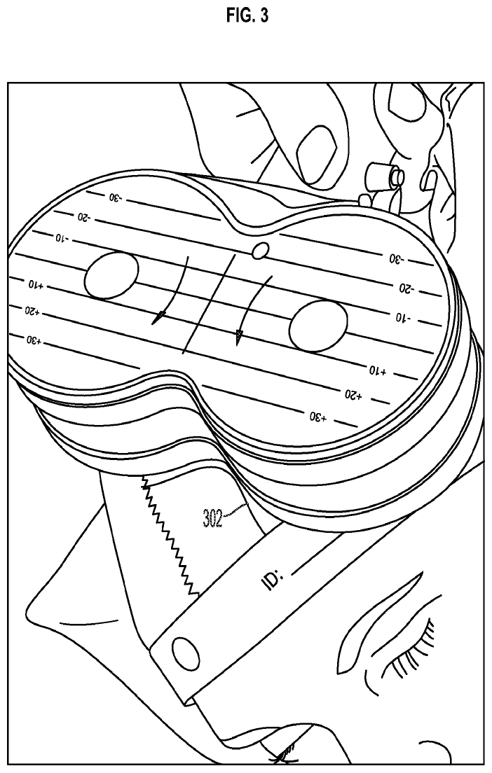

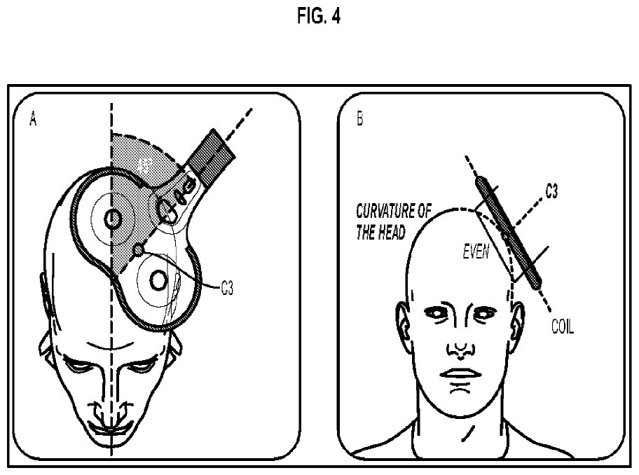

FIGS. 3 and 4 show an example MS coil apparatus as it may be used in practice. The MS coil apparatus 100 may be used in conjunction with a cap (e.g., resembling a swimmer's cap), that is fit snuggly over the patient's head. A target may be marked on the cap, and the MS coil may be centered over the target at the start of each treatment. The MS coil apparatus may be configured to project a reference line 302 which may be used by an operator to align an edge of the MS coil apparatus and thereby avoid visual parallax. The reference line 302 may be projected by a laser. FIG. 4 shows an example diagram of how the MS coil apparatus may be used in practice. (In this figure, C3 refers to a location on an EEG coordinate system that overlies part of the motor cortex.)

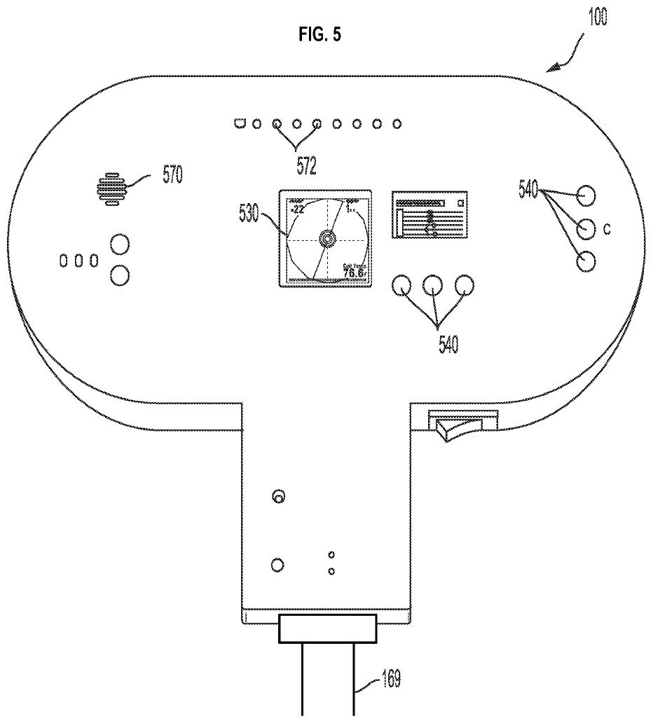

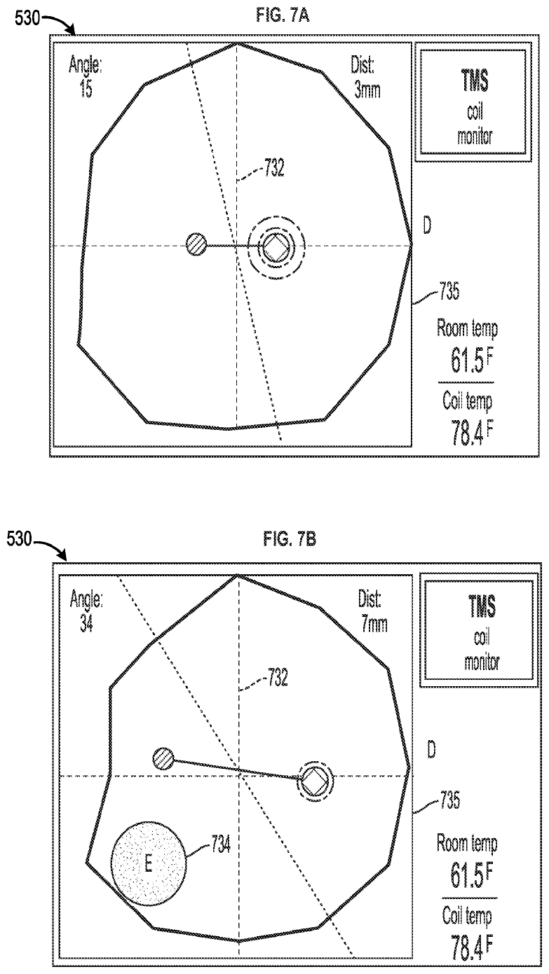

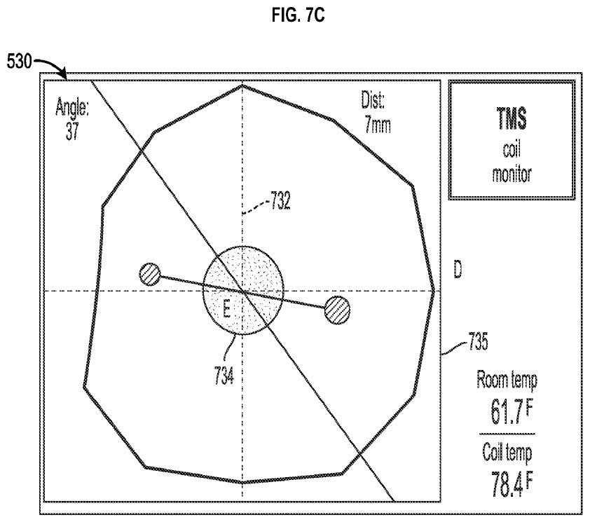

FIG. 5 shows an example MS coil apparatus 100. The MS coil apparatus 100 may comprise a display 530. The display 530 may be configured to output the MS data and/or MS alignment data received from the MS processing module 238. For example, the display 530 may be configured to display, based on at least one capacitive sensor of the one or more capacitive sensor 110, if any, are contacting the target marker 310, the position of the one or more capacitive sensors 110 with respect to the target marker 310. The display 530 will be described further herein with reference to FIGS. 7A and 7B. The MS coil apparatus 100 may comprise one or more buttons 540. Optionally, the one or more buttons 540 may be configured to receive an input (e.g., a press) and thereby toggle between what is shown on the display 530. The MS coil apparatus 100 may comprise a speaker 570. The speaker 570 may be configured to output one or more audio signals. For example, the speaker 570 may be configured to output the one or more audio signal when the MS coil device 100 is properly aligned. In an embodiment, speaker 570 can provide an indication that the coil temperature has passed a threshold. The MS coil apparatus 100 may comprise one or more alarm lights 572. The one or more alarm lights 572 may comprise one or more light emitting diodes (“LEDs”). The one or more alarm lights 572 may comprise, for example, red, green, blue LEDs (“RGB LEDs”). The one or more alarm lights 572 may be configured to indicate various conditions (e.g., whether the MS coil apparatus is centered on the target, tangential to the patient's head, and properly angled with respect to the mid-sagittal plane) based on, for example, which lights are lit, a number of lights that are lit, the intensity of the lights, and the color of the lights. The one or more alarm lights may be configured to provide feedback (e.g., indications) upon various conditions such as the coil temperature satisfying a threshold.

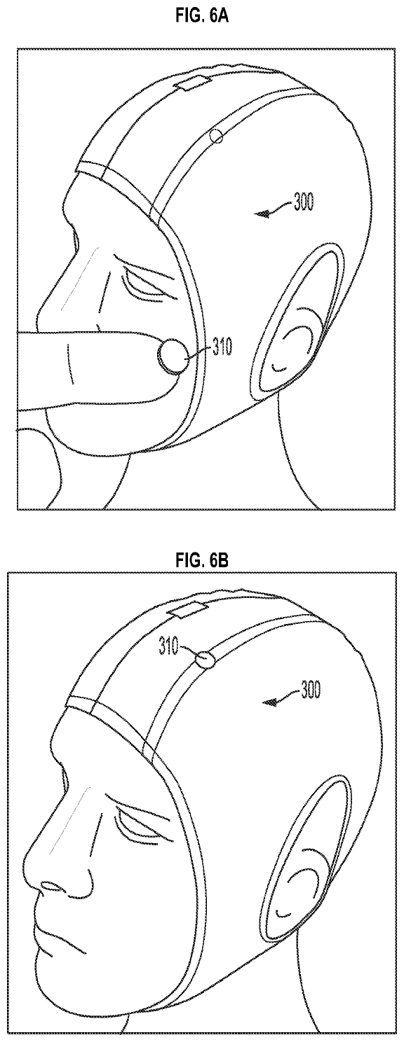

FIGS. 6A and 6B, show an example operating environment in which the MS coil apparatus may be used. For example, the MS coil apparatus 100 may cooperate with a cap 300 that is placed over the patient's head. The cap 300 may be thin and may be configured to fit on the patient's head. The cap 300 can be positioned with respect to fixed features on the patient's head (e.g., eyes, nose, ears) so that the cap 300 can be repeatedly placed on and removed from the patient's head in the same position. Optionally, the cap 300 can be marked with respect to the fixed features on the patient's head so that the cap 300 can be repeatedly placed on and removed from the patient's head in the same alignment. A target marker 310 can be placed on the cap 300 at the intended area of stimulation. The target marker 310 can be circular (e.g., ½ centimeter in diameter) and thin to minimize spacing of the MS coil from the patient's head. The target marker 310 may comprise a conductive material. The target marker may have an adhesive back configured to attach the marker to the cap 300. It is to be understood that the target marker 310 may be placed anywhere on the cap 300 and/or the patient's head.

The MS coil apparatus 100 can be placed against the patient's head so that the at least one capacitive sensor of the one or more capacitive sensors 110 rests against the target marker 310. The target marker 310 can contact and/or be placed in proximity to the one or more capacitive sensors 110 such that the target marker 310, being conductive, will create an increase in capacitance when placed in close proximity to a capacitive sensor electrode. It may be determined which of the one or more conductive sensors is contacting the target marker 310. For example, the controller can continuously or intermittently poll to detect which at least one capacitive sensor of the one or more capacitive sensors 110 is/are contacting the target marker 310. In this way, the controller can determine the position of the MS coil apparatus 100 with respect to the target marker 310 and, thus, the intended area of stimulation.

FIGS. 7A and 7B show an example display 530. The display 530 may comprise any display configured to output (e.g., display) any data. For example, the display 530 may comprise an LCD screen. The display 530 may be configured to show which capacitive sensor(s) of the one or more capacitive sensors 110, if any, are contacting the target marker 310. The display 530 may be configured to display a location of the one or more capacitive sensors 110 in relation to the target marker 310. In at least one embodiment, the display 530 can show a spot 734 in relation to a crosshair 732. The display 530 may be configured output data configured to indicate a position of the TMS coil apparatus 100. For example, the display 530 may be configured to indicate a location of the one or more capacitive sensors 110 contacting the target marker 310.

For example, a location of the spot 734 on the display in relation to the center of the crosshair 732 may indicate which capacitive sensor of the one or more capacitive sensors 110, if any, are in contact with the target marker. For example, in FIG. 7A, the display 530 does not show the spot 734, indicating that no contacts pads of the one or more capacitive sensors 110 are contacting the target marker 310. In FIG. 7B, the spot 734 is spaced from the center of the crosshair 732, indicating that the MS coil apparatus 100 is not aligned tangential to the patient's head with the axis of the coil directed towards the target area of the target surface.

FIG. 7C illustrates the spot 734 in the center of the crosshair 732, indicating that the axis of the MS coil apparatus 100 is centered over the target marker 310 and, thus, the target area of the target surface. In this way, an operator can receive feedback via the display 530 and move the MS coil apparatus until the MS coil is properly positioned with respect to the target area. Optionally, the display 530 can show a border 735 that changes color (e.g., from red to green) when the MS coil apparatus 100 contacts the target marker 310.

It should be understood that the display 530 can show an average position of the capacitive sensor of the one or more capacitive sensors 110 that are contacting the target marker 310. For example, if two capacitive sensors 110 are touching the target marker 310 simultaneously, the display 530 can show the spot 734 in a location with respect to the crosshairs 732 corresponding to the position between the two capacitive sensors 110. As another example, if six capacitive sensors 110 arranged in a 2×3 grid are touching, the display can display the location corresponding to the position of the 2×3 grid's center.

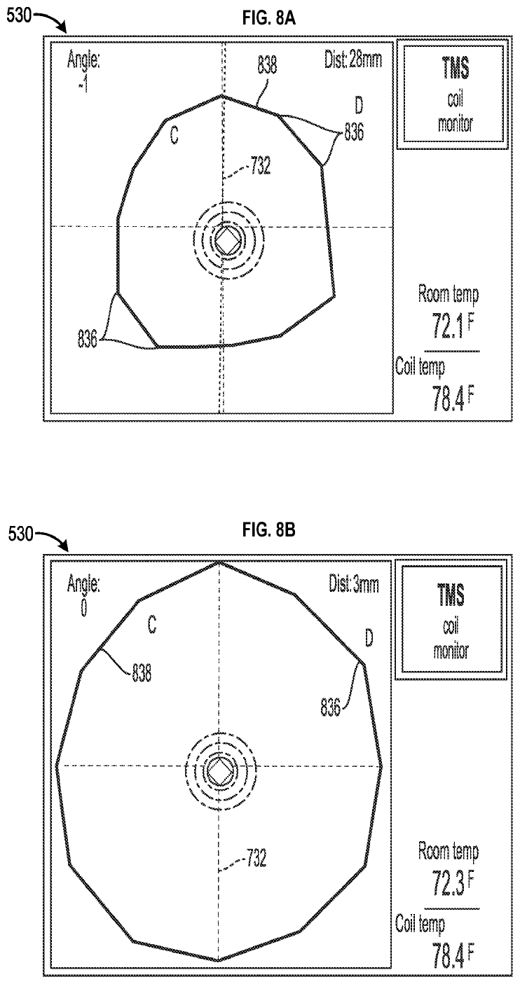

FIGS. 8A and 8B show the display 530. The display 530 can be configured to display the respective distances of each range sensor of the plurality of range sensors 120 as one or more vertex 836 of a polygon 838, wherein the distance from the vertex 836 to the center of the crosshair 732 indicates (e.g., corresponds to) the distance from a range sensor of the one or more range sensors to the patient's head (e.g., the target surface). Optionally, the distance from the center of the crosshair 732 to each vertex can increase or decrease as a respective distance between the range sensor of the plurality of range sensors 120 and the target surface increases or decreases. Accordingly, an operator can orient the MS coil apparatus 100 to center the polygon 838 with the center of the crosshair 732, indicating that the MS coil apparatus 100 is generally tangential to the patient's head.

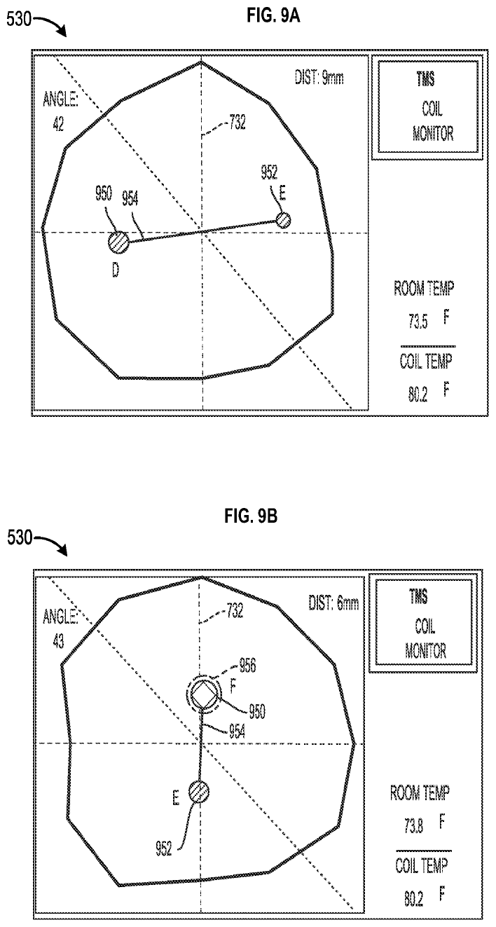

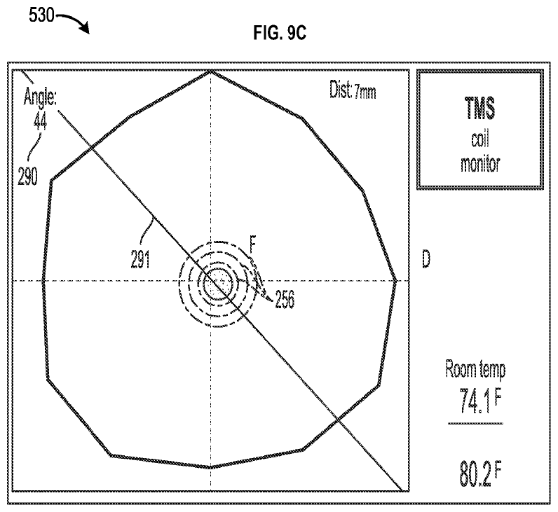

FIGS. 9A-9C show further examples of the display 530. For example, the controller can aggregate data from the plurality of range sensors 120 to provide an indication to the operator as to how close the MS coil apparatus 100 is to tangential to the patient's head as well as how the MS coil apparatus 100 can be adjusted to further improve its tangential orientation. For example, the controller can compute an average of the vectors corresponding to each range sensor's the distance to the patient's head and the range sensor's respective azimuthal angle about the MS coil's axis. For example, the controller can compute a vector sum based on distance measurements and the position of each range sensor of the plurality of range sensors 120. The relative distances (e.g., vectors) may be compared to an angle of the coil with respect to the head. For example, if a range sensor located at the 12 o'clock position is closer to the head than a range sensor the 6 o'clock position, it may be determined that the coil is tiled with respect to the head. The display 530 can then show a first spot 950 indicating a vector based on the sum of the range sensors' vectors. The first spot 950 can be spaced from the center of the crosshair 732 showing a direction toward which the MS coil apparatus 100 should be tilted to improve the tangential orientation.

The display 530 can further show a complementary second spot 952 equally spaced in the opposite direction from the center of the crosshair 732 and a line 954 between the first spot 950 and the second spot 952, showing a visible vector further indicating the direction that the MS coil apparatus 100 should be tilted to be property aligned with respect to the target area of the target surface (e.g., contacting the marker 310 and tangential to the surface of the patient's head). The display 530 can optionally show the average distance 958 from the plurality of range sensors 120 to the patient's head.

As the first spot 950 and the second spot 952 converge, it should be understood that they may overlap. As the first spot 950 nears the crosshair's center, the display 530 can show an increasing number of concentric rings 956 around the first spot 950. For example, zero concentric rings 956 (FIG. 9A) can show that the MS coil apparatus 100 is far from tangential alignment, one concentric ring (FIG. 9B) can indicate better tangential alignment, two concentric rings (not shown) can indicate still better tangential alignment, and three concentric rings (FIG. 9C) can indicate optimal tangential alignment. The display 530 can show the MS coil apparatus's orientation with respect to the reference orientation as a numerical display 290 and geometrically as a line 291 angled with respect to the crosshairs 732.

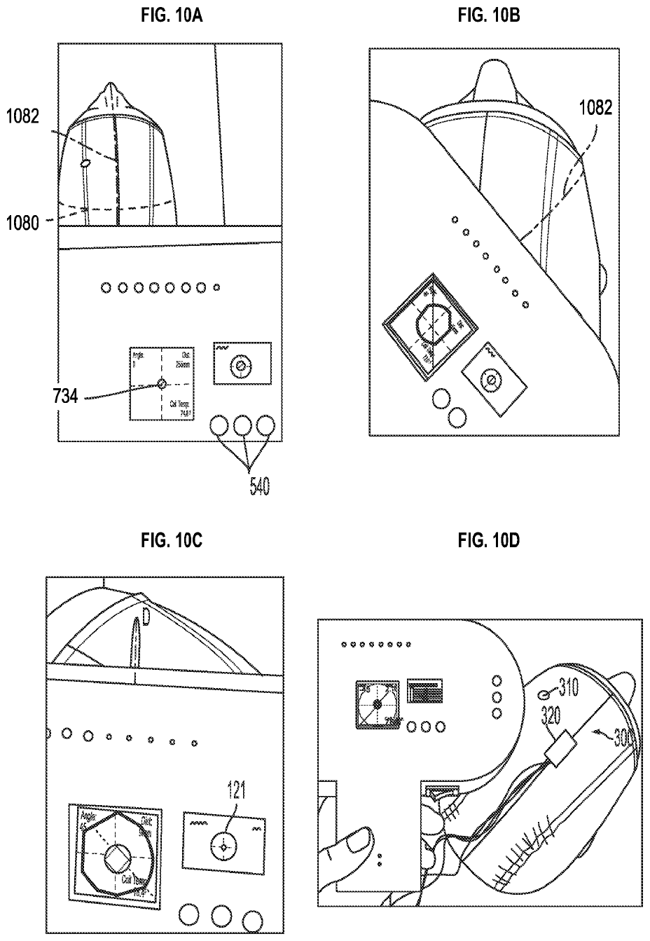

FIGS. 10A-10D show one or more MS coil apparatuses 100. For example, optionally, the one or more buttons 540 can toggle between what is shown on the display. Accordingly, in at least one embodiment, the operator can switch between the display showing the spot 734 and the first and second spots 950, 952. In further embodiments, the display can show all of spot 734 and first and second spots 950, 952 simultaneously. For example, the MS coil apparatus 100 can further comprise one or more laser guides and an absolute 3-axis orientation sensing system. According to some aspects, the MS coil apparatus 100 can comprise a vertical axis laser 1080 and a horizontal axis laser 1082 that can each comprise a laser diode. The vertical axis laser 1080 can emit a first laser line that is aligned with the MS coil's axis, and the horizontal laser 1082 can emit a second laser line that is perpendicular to the first laser line and is aligned with the MS coil's axis. Accordingly, the first and second laser lines can intersect at (e.g., a location in the patient's head corresponding to) the stimulation site. The laser lines can optionally be turned on and off. In various embodiments, the MS coil apparatus 100 can comprise a plurality of LEDs spaced circularly around the display 530 that can indicate (e.g., based on their colors, intensity, and on/off status) a direction that the MS coil apparatus should be moved, how it should be tilted, and/or how it should be rotated in order to properly align the MS coil apparatus 100 with respect to the target area of the target surface. In an embodiment, based on the information from the sensors, specification instructions may be determined which may be displayed so as to aid in the placement of the coil. For example, an instruction such as “move coil left,” or “tilt coil forward,” or “turn coil clockwise,” may be displayed. Such instructions may be conveyed via text, audio outputs, or visual displays such as graphical displays.

The operator can align the first laser line 1082 with a vertical line on the cap 300. Once the laser line and the vertical line are aligned (corresponding with the MS coil being at a zero-degree alignment with respect to the mid-sagittal plane), the operator can then actuate a button of the one or more buttons 540 that causes the MS coil apparatus 100 to store the three-dimensional orientation information that can be used as a reference orientation. Accordingly, as it can be desirable to orient the MS coil apparatus 100 at forty-five degrees with respect to the mid-sagittal plane, the MS coil apparatus 100 can be tilted forty-five degrees from the reference orientation with respect to the mid-sagittal plane for optimal results.

A reference orientation sensor module 320 (as seen in FIG. 10D) can be secured to the patient (e.g., the cap 300), for example, as a component that is integral to the cap 300 or attached to the cap 300 with a fastener such as hook and loop. The reference orientation sensor module 320 can comprise orientation sensors (e.g., accelerometers, gyroscopes, magnetometers, combinations thereof, and the like) and act as a reference for comparing movement of the MS coil apparatus 100 with movement of the patient. In this way, after alignment, the operator can determine if movement of the MS coil apparatus 100 corresponds with respective movement of the patient or if the MS coil apparatus 100 has moved with respect to the patient. For example, if the reference orientation sensor module 320 detects that the patient moves his or her head five degrees about an axis, and the orientation sensors 150 detect that the MS coil apparatus similarly rotates five degrees about the axis, it can be understood that the MS coil apparatus is still likely in proper alignment with the target area. If, however, the orientation sensors 150 detect that MS coil apparatus rotates five degrees about the axis, and the reference orientation sensor module 320 detects no movement, it can be understood that the MS coil likely moved with respect to the patient and is no longer in proper alignment with the target area.

Referring to FIG. 10C, optionally, a secondary display 121 can provide various information to the operator, including a virtual “bubble level” that can show orientation with respect to a pair of axes. The operator can optionally set the reference positions for said pair of axes. The virtual bubble level can be used for orienting the coil. In some embodiments, the virtual bubble levels can provide graphic representations of accelerometer data either in absolute readings or compared to a reference orientation. In further embodiments, the virtual bubble levels can provide graphic representation of a relationship between the MS coil apparatus 100 and the reference orientation sensor module 320. In still further embodiments, the virtual bubble levels can illustrate orientation changes with respect to a treatment orientation—that is, with respect to orientation data captured at a previous time when the MS coil apparatus 100 was positioned for a treatment.

FIG. 10D illustrates an example image of dynamic reference orientation sensor 320 that is attached to a patient for providing orientation data of the patient for comparison to orientation data from the MS coil apparatus.

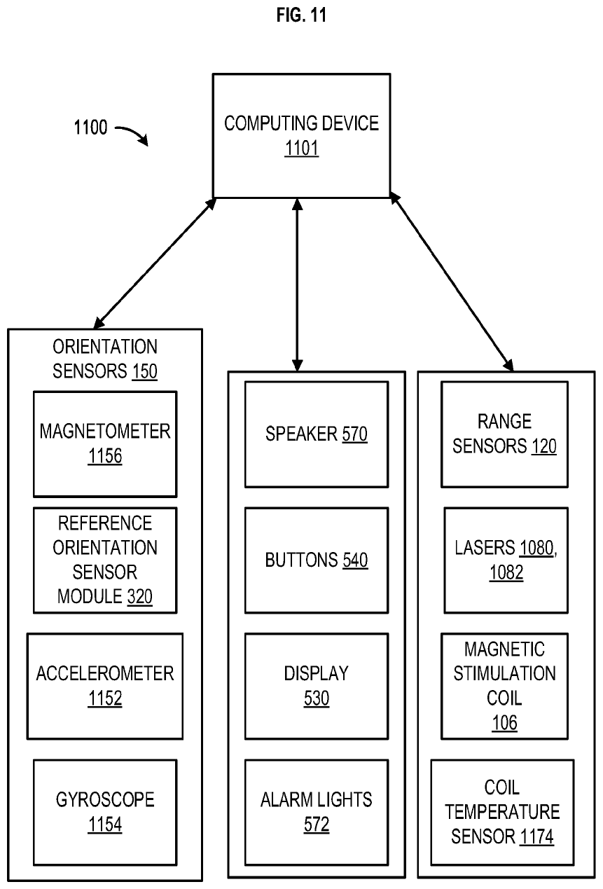

FIG. 11 shows a schematic diagram of the MS coil apparatus 100 system 1100. The MS coil apparatus 100 may can comprise orientation sensors such as, for example, a three-axis accelerometer 1152, a three-axis gyroscope 1154, the reference orientation sensor module 320 and/or a three-axis magnetometer 1156. The system may comprise an MS coil 106. The three-axis accelerometer 1152 can optionally comprise a plurality of accelerometers that are oriented with respect to each other to sense orientation along their respective axes so that their cumulative orientation data can cooperate to provide the orientation of the three-axis accelerometer 1152 in all three dimensions.

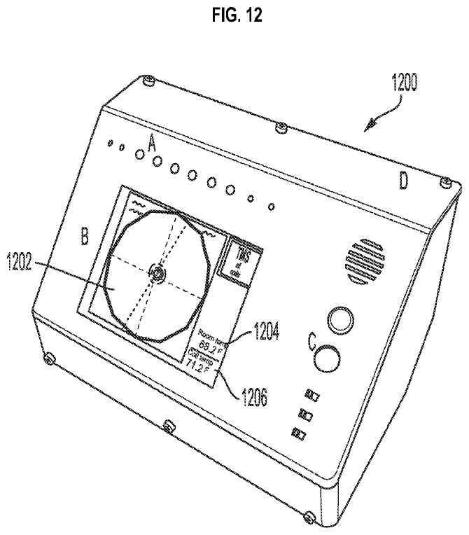

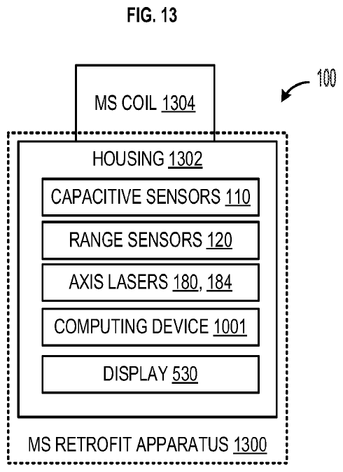

The MS coil apparatus 100 and/or remote display 1200 can further include the speaker 570 to give audio feedback to the operator. For example, the computing device 1101 can determine that the MS coil apparatus has moved a threshold amount from its alignment with the target. In response, the MS coil apparatus can provide audible feedback to the operator. Further, the MS coil apparatus 100 and/or the remote display 1200 (with reference to FIG. 12) can comprise lights 572 (e.g., RGB LEDs). The lights 572 may configured to indicate various conditions (e.g., whether the MS coil apparatus is centered on the target, tangential to the patient's head, and properly angled with respect to the mid-sagittal plane) based on, for example, which lights are lit, a number of lights that are lit, the intensity of the lights, and the color of the lights. For example, the LEDs may be configured to indicate a direction or change in position required to properly align the MS coil apparatus 100. For example, the lights 572 may be configured to indicate (e.g., by turning on or off in series or changing color or intensity) that the MS coil apparatus 100 should be titled right or left while the LEDs may be configured to indicate the MS coil apparatus should be tilted forward or back or any other direction. A computing device 1001 (as seen in FIG. 13) can be programmed for providing feedback with the speaker 570 and lights 572 upon various conditions. In some embodiments, the MS coil apparatus 100 can have a coil temperature sensor 1174. The speaker 570 and/or lights 572 can provide an indication that the coil temperature has passed a threshold.

It is common during MS operations that the operator is several feet away from the patient and thus the display 530 may not be viewable to the operator. Moreover, MS operations can typically take between a few minutes and an hour, during which the patient may move. Although the MS coil apparatus 100 may be locked into place and secured by an articulated support arm that is commonly used in the art, small movements of the patient can result in disrupted engagement of the target with the MS coil apparatus 100, which can lead to less than optimal results. Accordingly, the MS coil apparatus 100 can include a wireless transmitter in communication with a remote display 1200 that can provide real-time positioning information that is similar to that of the display 530. The articulated support may be configured to adjust the MS coil apparatus 100 to maintain optimal alignment. In an example, the articulated support arm may be guided by software that receives real-time data from the MS coil apparatus 100 indicating the alignment of the MS coil apparatus with respect to the target area of the target surface (e.g., the marker 310).

FIG. 12 shows an example remote display 1200. For example, the remote display 1200 can include an LED, OLED, LCD, or other graphic display 1202. The remote display 1200 can show all of the same information as display 530, including the crosshair 732 and the spot 734, the polygon 836 and the first and second spots 950, 952, and line 954. The remote display 1200 can further show the numerical display the line angled with respect to the crosshairs.

Accordingly, the remote display 1200 can show all of the information provided on the display 530. Moreover, the remote display 1200 can show additional information, such as room temperature 1204, which can contribute to coil overheating, and coil temperature 1206.

Accordingly, the MS coil apparatus 100, as disclosed herein enable an operator to align the MS coil with the target. In particular, the MS coil apparatus 100 can provide the operator with visual feedback (including the display 530, the vertical axis laser 1080, and the horizontal axis laser 1084) for properly aligning the MS coil.

FIG. 13 shows, components of the MS coil apparatus 100. The MS coil apparatus 100 can be configured to retrofit an MS coil 1304. For example, in one embodiment, an MS retrofit apparatus 1300 can comprise a housing 1302. The housing 1302 can couple to the MS coil 1304 in a fixed location with respect to the MS coil's stimulation axis. The housing 1302 can be configured to be removably attached to and/or detached from a MS coil e.g., via clips or hook and loop fasteners, snaps, magnets, etc. Preferably, the housing 1302 can be configured to cause minimal additional spacing in the axial dimension from the patient's head. Accordingly, the housing 1302 can comprise a thin material. The MS retrofit apparatus 1300 can comprise some or all of the features of the MS coil apparatus 100, including, but not limited to: the one or more capacitive sensors 110, a plurality of range sensors 120, vertical and horizontal axis lasers 1080, 1084, the computing device 1001, and a display 530, wireless transceiver, and/or other output devices.

The MS coil apparatus 100 can be used according to the following method. The cap 300 can be placed on the patient and aligned with respect to the patient's head/facial features. The target marker 310 can be placed on the cap 300 at the desired target for stimulation. An operator can, using the vertical and horizontal axis lasers 180, 182, orient the MS coil apparatus to get a zero-degree reference orientation from which the forty-five degree orientation from the mid-sagittal plane is determined. The operator can then rotate the MS coil apparatus to the desired forty-five degree orientation. Then, the operator can, using feedback from the display 530 showing engagement between at least one capacitive sensor of the one or more capacitive sensors 110 and the target marker 310, position the MS coil apparatus 100 so that the MS coil apparatus 100 is centered at the target. The operator can then pivot the MS coil apparatus 100 until it is tangentially aligned with the patient's head, adjusting the position as needed so that the MS coil is still centered at the target. The operator can begin using the MS coil to stimulate the target area while using the remote display 1200 to continually monitor the orientation and other aspects (e.g., coil temperature) from a remote location.

In some embodiments, the MS coil apparatus 100 and, in particular, one or more capacitive sensors 110 and target marker 310, can be used to identify a target area such as a motor hotspot. A motor hotspot can be an area over the patient's motor cortex. Conventionally, the MS intensity is varied until it reliably induces a twitch of the patient's contralateral abductor pollicus brevis muscle. This can be a time-consuming process. By marking the hotspot with a target marker 310, the motor threshold can be assessed quickly and reliably.

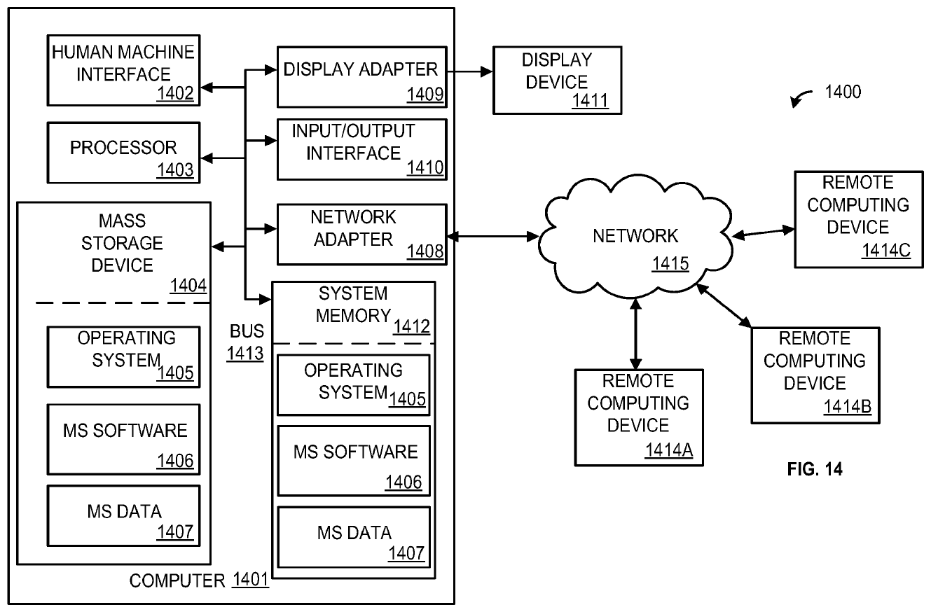

FIG. 14 shows a system 1400 including a computing device 1401 for use with the MS coil apparatus 100.

The computing device 1401 may comprise one or more processors 1403, a system memory 1412, and a bus 1413 that couples various components of the computing device 1401 including the one or more processors 1403 to the system memory 1412. In the case of multiple processors 1403, the computing device 1401 may utilize parallel computing.

The bus 1413 may comprise one or more of several possible types of bus structures, such as a memory bus, memory controller, a peripheral bus, an accelerated graphics port, and a processor or local bus using any of a variety of bus architectures.

The computing device 1401 may operate on and/or comprise a variety of computer readable media (e.g., non-transitory). Computer readable media may be any available media that is accessible by the computing device 1401 and comprises, non-transitory, volatile and/or non-volatile media, removable and non-removable media. The system memory 1412 has computer readable media in the form of volatile memory, such as random access memory (RAM), and/or non-volatile memory, such as read only memory (ROM). The system memory 1412 may store data such as MS data 1407 and/or program modules such as operating system 1405 and MS software 1406 that are accessible to and/or are operated on by the one or more processors 1403.

The computing device 1401 may also comprise other removable/non-removable, volatile/non-volatile computer storage media. The mass storage device 1404 may provide non-volatile storage of computer code, computer readable instructions, data structures, program modules, and other data for the computing device 1401. The mass storage device 1404 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1404. An operating system 1405 and MS software 1406 may be stored on the mass storage device 1404. One or more of the operating system 1405 and MS software 1406 (or some combination thereof) may comprise program modules and the MS software 1406. MS data 1407 may also be stored on the mass storage device 1404. MS data 1407 may be stored in any of one or more databases known in the art. The databases may be centralized or distributed across multiple locations within the network 1415.

A user (e.g., the clinician) may enter commands and information into the computing device 1401 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices may be connected to the one or more processors 1403 via a human machine interface 1402 that is coupled to the bus 1413, but may be connected by other interface and bus structures, such as a parallel port, game port, an IEEE 1394 Port (also known as a Firewire port), a serial port, network adapter 1408, and/or a universal serial bus (USB).

A display device 1411 may also be connected to the bus 1413 via an interface, such as a display adapter 1409. It is contemplated that the computing device 1401 may have more than one display adapter 1409 and the computing device 1401 may have more than one display device 1411. A display device 1411 may be a monitor, an LCD (Liquid Crystal Display), light emitting diode (LED) display, television, smart lens, smart glass, and/or a projector. In addition to the display device 1411, other output peripheral devices may comprise components such as speakers (not shown) and a printer (not shown) which may be connected to the computing device 1401 via Input/Output Interface 1410. Any step and/or result of the methods may be output (or caused to be output) in any form to an output device. Such output may be any form of visual representation, including, but not limited to, textual, graphical, animation, audio, tactile, and the like. The display 1411 and computing device 1401 may be part of one device, or separate devices.

The computing device 1401 may operate in a networked environment using logical connections to one or more remote computing devices 1414A,B,C. A remote computing device 1414A,B,C may be a personal computer, computing station (e.g., workstation), portable computer (e.g., laptop, mobile phone, tablet device), microcontroller, smart device (e.g., smartphone, smart watch, activity tracker, smart apparel, smart accessory), security and/or monitoring device, a server, a router, a network computer, a peer device, edge device or other common network node, and so on. Logical connections between the computing device 1401 and a remote computing device 1414A,B,C may be made via a network 1415, such as a local area network (LAN) and/or a general wide area network (WAN). Such network connections may be through a network adapter 1408. A network adapter 1408 may be implemented in both wired and wireless environments. Such networking environments are conventional and commonplace in dwellings, offices, enterprise-wide computer networks, intranets, and the Internet.

Application programs and other executable program components such as the operating system 1405 are shown herein as discrete blocks, although it is recognized that such programs and components may reside at various times in different storage components of the computing device 1401, and are executed by the one or more processors 1403 of the computing device 1401. An implementation of MS software 1406 may be stored on or sent across some form of computer readable media. Any of the disclosed methods may be performed by processor-executable instructions embodied on computer readable media.

In some embodiments, the computing device 1401 may be electronically connected to one or more imaging devices, for example a device or system for performing one or more of computed tomography, radiography, medical resonance imaging, or ultrasound.

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An apparatus comprising: a magnetic stimulation coil having a central axis; one or more capacitive sensors, wherein the one or more capacitive sensors is configured to contact a target on a target area of a target surface; processing circuitry configured to detect an engagement between the conductor and at least of the one or more capacitive sensors; the plurality of range sensors 120 spaced from the central axis of the magnetic stimulation coil; and a display configured to display: a location corresponding to the engagement between the conductor and the at least one capacitive sensors, distance between each range sensor and the target surface, and angle of coil rotation with respect to a reference position . . .

Aspect 2: The apparatus of aspect 1, further comprising a first light emitting device configured to display a first line, and a second light emitting device configured to display a second line.

Aspect 3: The apparatus of aspect 2, wherein each of the first light emitting device and the second light emitting device is a laser diode.

Aspect 4: The apparatus of any of the previous aspects, further comprising at least one orientation sensor, wherein the processing circuitry is configured to determine an orientation of the magnetic stimulation coil apparatus based on data from the at least one orientation sensor.

Aspect 5: The apparatus of aspect 4, wherein the at least one orientation sensor comprises a three-axis accelerometer, a three-axis gyroscope, and a three-axis magnetometer.

Aspect 6: The apparatus of aspect 4 or aspect 5, further comprising memory, wherein the processing circuitry is configured to store in the memory a reference orientation based on at least one measurement from the at least one orientation sensor, wherein the processor is configured to determine a relative orientation of the MS coil with respect to the reference orientation, wherein the display is further configured to display the relative orientation of the MS coil apparatus with respect to the reference orientation.

Aspect 7; The apparatus of any of the previous aspects, wherein each electrical contact is evenly spaced from each respective adjacent electrical contact.

Aspect 8: The apparatus of any of the previous aspects, wherein the plurality of range sensors comprises at least two range sensors.

Aspect 9: The apparatus of any of the previous aspects, further comprising a wireless transmitter that is configured to transmit data captured by the apparatus to a receiver operably coupled to a remote display.

Aspect 10: The apparatus of any of the previous aspects, wherein the one or more capacitive sensors is centered with respect to the central axis, and the plurality of range sensors are equally spaced from the central axis.

Aspect 11: The apparatus of any of the previous aspects, wherein the one or more capacitive sensors are configured in one or more rows or columns.

Aspect 12: The apparatus of any of the previous aspects, further comprising a second orientation sensor, wherein the processing circuitry is configured to compare orientation data from the second orientation sensor to orientation data from the at least one orientation sensor.

Aspect 13: An apparatus comprising: a housing configured to couple to a magnetic stimulation coil device; one or more capacitive sensors, wherein the one or more capacitive sensors is configured to contact a target on a target area of a target surface; and processing circuitry configured to detect an engagement between the conductor and at least one capacitive sensor of the one or more capacitive sensors; and a plurality of range sensors disposed around a circumference of the one or more capacitive sensors.

Aspect 14: The apparatus of aspect 13, further comprising a display that is configured to display: a location corresponding to the engagement between the conductor and the at least one capacitive sensor, and a distance between each range sensor and the target surface.

Aspect 15: The apparatus of aspect 14, further comprising: at least one orientation sensor, wherein the processing circuitry is configured to determine an orientation of the magnetic stimulation coil apparatus based on data from the at least one orientation sensor; and memory, wherein the processing circuitry is configured to store in the memory a reference orientation based on at least one measurement from the at least one orientation sensor, wherein the processor is configured to determine a relative orientation of the MS coil with respect to the reference orientation, wherein the display is further configured to display the relative orientation of the MS coil apparatus with respect to the reference orientation.

Aspect 16: The apparatus of any of aspects 13-15, further comprising a wireless transmitter that is configured to transmit data captured from the apparatus to a receiver operably coupled to a remote display.

Aspect 17: A system comprising: an apparatus comprising: a magnetic stimulation coil having a central axis; one or more capacitive sensors, wherein the one or more capacitive sensors is configured to contact a target on a target area of a target surface; processing circuitry configured to detect an engagement between the conductor and at least one electrical contact of the one or more capacitive sensors; a plurality of range sensors spaced from the central axis of the magnetic stimulation coil; and a wireless transmitter; and a remote display comprising a receiver and that is configured to display: a location corresponding to the engagement between the conductor and the at least one electrical contact, distance between each range sensor and the target surface, and angle of coil rotation with respect to a reference position, wherein the wireless transmitter is configured to transmit data captured by the apparatus to the receiver of the remote display.

Aspect 18: The system of aspect 17, wherein the apparatus further comprises at least one orientation sensor, wherein the processing circuitry is configured to determine an orientation of the magnetic stimulation coil apparatus based on data from the at least one orientation sensor, wherein the remote display comprises memory, wherein the processing circuitry is configured to store a reference orientation based on at least one measurement from the at least one orientation sensor, wherein the processor is configured to determine a relative orientation of the MS coil with respect to the reference orientation, wherein the display is further configured to display the relative orientation of the MS coil apparatus with respect to the reference orientation.

Aspect 19: A method comprising: receiving a signal corresponding to an engagement between a conductor and at least a first electrical contact and a second electrical contact of one or more capacitive sensors; determining, based on the signal, a contact location, wherein the contact location is a position between the first electrical contact and the second electrical contact; receiving a distance measurement from each of a plurality of range sensors; displaying the contact location on a display; and displaying the distance measurement from each of the plurality of range sensors on the display.

Aspect 20: The method of aspect 19, wherein displaying the contact location on the display comprises graphically displaying the contact location as a radial offset from a center point.

Aspect 21: The method of aspect 19 or aspect 20, wherein displaying the distance measurement from each of the plurality of range sensors on the display comprises graphically displaying the distance measurement from each of the plurality of range sensors as a radius from a center point.

Aspect 22: The method of any of aspects 19-21, further comprising: calculating a vector as a function of each distance measurement from each of the plurality of range sensors; and displaying the vector on the display.

Aspect 23: The method of any of aspects 19-22, further comprising transmitting the signal and the contact location to the display, wherein the display is a remote display.

Although the foregoing invention has been described in some detail by way of illustration and example for purposes of clarity of understanding, certain changes and modifications may be practiced within the scope of the appended claims.

What is claimed is:

1. An apparatus, comprising:
one or more processors;
a magnetic stimulation coil;
one or more capacitive sensors, wherein at least one capacitive sensor of the one or more capacitive sensors is configured to contact a target surface;
one or more range sensors spaced from the central axis of the magnetic stimulation coil;
memory storing processor executable instructions that, when executed by the one or more processors, cause the apparatus to detect contact between the at least one capacitive sensor and the target surface; and
a display configured to display:
a location associated with the contact between the at least one capacitive sensor and the target surface, and
a distance between at least one range sensor of the one or more range sensors and the target surface.

2. The apparatus of claim 1, further comprising a first light emitting device configured to display a first line, and a second light emitting device configured to display a second line.

3. The apparatus of claim 2, wherein each of the first light emitting device and the second light emitting device is a laser diode.

4. The apparatus of claim 1, further comprising at least one orientation sensor, wherein the processing circuitry is configured to determine an orientation of the apparatus based on data from the at least one orientation sensor.

5. The apparatus of claim 4, wherein the at least one orientation sensor comprises a three-axis accelerometer, a three-axis gyroscope, and a three-axis magnetometer.

6. The apparatus of claim 4, further comprising memory, wherein the processing circuitry is configured to store in the memory a reference orientation based on at least one measurement from the at least one orientation sensor, wherein the processing circuitry is configured to determine a relative orientation of the magnetic stimulation (MS) coil with respect to the reference orientation, wherein the display is further configured to display the relative orientation of the MS coil with respect to the reference orientation.

7. The apparatus of claim 1, wherein each capacitive sensor is evenly spaced from each respective adjacent capacitive sensor.

8. The apparatus of claim 1, wherein the plurality of range sensors comprises at least two range sensors.

9. The apparatus of claim 1, further comprising a wireless transmitter configured to transmit data captured by the apparatus to a receiver operably coupled to a remote display.

10. The apparatus of claim 1, further comprising one or more capacitive sensors, wherein the one or more electrical contents is centered with respect to the central axis, and the plurality of range sensors are equally spaced from the central axis.

11. The apparatus of claim 1, wherein the one or more capacitive sensors are configured in one or more rows or one or more columns.

12. The apparatus of claim 4, further comprising a second orientation sensor, wherein the processing circuitry is configured to compare orientation data from the second orientation sensor to orientation data from the at least one orientation sensor.

13. An apparatus, comprising:
one or more processors;
a housing configured to couple to a magnetic stimulation coil;
one or more capacitive sensors, wherein at least one capacitive sensor of the one or more capacitive sensors is configured to contact a target surface;
memory storing processor executable instructions that, when executed by the one or more processors, cause the apparatus to detect the contact between the at least one capacitive sensor and the target surface; and
one or more range sensors disposed around a circumference of the one or more capacitive sensors.

14. The apparatus of claim 13, further comprising a display configured to display:
a location corresponding to the contact between the at least one capacitive sensor and the target surface; and
a distance between the at least one range sensor and the target surface.

15. The apparatus of claim 14, further comprising:
at least one orientation sensor, wherein the processing circuitry is configured to determine an orientation of the magnetic stimulation coil based on data from the at least one orientation sensor; and
memory, wherein the processing circuitry is configured to store in the memory a reference orientation based on at least one measurement from the at least one orientation sensor, wherein the processing circuitry is configured to determine a relative orientation of the magnetic stimulation coil with respect to the reference orientation, wherein the display is further configured to display the relative orientation with respect to the reference orientation.

16. The apparatus of claim 15, further comprising a wireless transmitter configured to transmit data captured from the apparatus to a receiver operably coupled to a remote display.

17. A system comprising:
an apparatus comprising:
a magnetic stimulation coil having a central axis;
one or more capacitive sensors, wherein at least one capacitive sensors of the one or more capacitive sensors is configured to contact a target surface;
processing circuitry configured to detect the contact between the at least one capacitive sensor and the target surface;
one or more range sensors spaced from the central axis of the magnetic stimulation coil;
a wireless transmitter; and
a display comprising a receiver, wherein the display is configured to display:
a location associated with the contact between the at least one capacitive sensor and the target surface, and
a distance between the at least one range sensor and the target surface,
wherein the wireless transmitter is configured to transmit data captured by the apparatus to the receiver of the display.

18. The system of claim 17, wherein the apparatus further comprises at least one orientation sensor, wherein the processing circuitry is configured to determine an orientation of the magnetic stimulation coil based on data from the at least one orientation sensor, wherein the remote display comprises memory, wherein the processing circuitry is configured to store a reference orientation based on at least one measurement from the at least one orientation sensor, wherein the processing circuitry is configured to determine a relative orientation of the magnetic stimulation coil with respect to the reference orientation, wherein the display is further configured to display the relative orientation of the magnetic stimulation coil with respect to the reference orientation.

19. The system of claim 17, further comprising a first light emitting device configured to display a first line, and a second light emitting device configured to display a second line.

20. The system of claim 19, wherein each of the first light emitting device and the second light emitting device is a laser diode.
